(19) United States
(12) Patent Application Publication
(10) Pub. No.: US 2020/0122131 A1
(43) Pub. Date: Apr. 23, 2020

(54) Methods of Producing Linear Alpha Olefins

(71) Applicant: SABIC GLOBAL TECHNOLOGIES B.V., RIYADH (SA)

(72) Inventors: Abdullah ALQAHTANI, Riyadh (SA); Sebastiano LICCIULLI, Riyadh (SA); Mohammed Fahad AL-ANAZI, Riyadh (SA)

(21) Appl. No.: 16/473,338

(22) PCT Filed: Dec. 28, 2017
(86) PCT No.: PCT/IB17/58484

§ 371 Date: Jun. 25, 2019

Related U.S. Application Data
(60) Provisional application No. 62/440,430, filed on Dec. 30, 2016.

(51) Int. Cl. B01J 31/18; C07C 2/34; B01J 31/02; B01J 31/14

(52) U.S. Cl. CPC B01J 31/1845; C07C 2/34; B01J 31/0239; B01J 2231/20; C07C 2531/12; C07C 2531/14; B01J 31/143

(57) ABSTRACT

A method of producing linear alpha olefins includes: preparing a solution A, comprising: introducing an organometallic compound and an organic ligand to a first vessel, wherein the first vessel is in fluid communication with a Schlenk line; and introducing a solvent to the first vessel via the Schlenk line; preparing a solution B separately from solution A, comprising: introducing an ammonium salt to a second vessel, wherein the second vessel is in fluid communication with a Schlenk line; and introducing an organoaluminum compound and a solvent to the second vessel via the Schlenk line; producing the linear alpha olefins by introducing solution A and solution B to an ethylene oligomerization reactor.

### BACKGROUND

Linear alpha olefins are olefins or alkenes with a chemical formula CₓH₂ₓ, distinguished from other mono-olefins with a similar molecular formula by linearity of the hydrocarbon chain and the position of the double bond at the primary or alpha position. There are a wide range of industrially significant applications for linear alpha olefins. For example, the lower carbon numbers, 1-butene, 1-hexene and 1-octene can be used as co-monomer in the production of polyethylene.

Linear alpha olefins can often be produced via the oligomerization of ethylene. This process presents many engineering challenges. For example, catalysts for the production of linear alpha olefins are commonly prepared by direct injection into an ethylene oligomerization reactor. A first catalyst component (chromium, zirconium, or titanium compound) and a second catalyst component (aluminum compound) are injected into the reactor via the same or different nozzles. However, due to the direct injection, the timing of the catalyst employment is greatly restricted and therefore, on-demand use of the catalyst is not possible. Another common catalyst preparation method involves the pre-mixing of different catalyst components in advance. The catalyst mixture is then stored and employed for on-demand use. However, this method requires that the mixture remain stable for extended periods of time. This limits the possible catalyst components that can be used. Catalyst mixtures with low stability levels and/or short lives are not compatible. Accordingly, this method excludes mixtures that require relatively short residency times prior to the ethylene oligomerization reaction.

Thus, there is a need for a method that can prepare an ethylene oligomerization catalyst without storage time restrictions or component stability restrictions, and can be used on-demand to produce linear alpha olefins.

### SUMMARY

Disclosed, in various embodiments, are methods of producing linear alpha olefins.

A method of producing linear alpha olefins comprises: preparing a solution A, comprising: introducing an organometallic compound and an organic ligand to a first vessel, wherein the first vessel is in fluid communication with a Schlenk line; and introducing a solvent to the first vessel via the Schlenk line; preparing a solution B separately from solution A, comprising: introducing an ammonium salt to a second vessel, wherein the second vessel is in fluid communication with a Schlenk line; and introducing an organoaluminum compound and a solvent to the second vessel via the Schlenk line; producing the linear alpha olefins by introducing solution A and solution B to an ethylene oligomerization reactor.

A method of producing linear alpha olefins, comprising: drying a first vessel and a second vessel in an oven at a temperature greater than or equal to 50° C.; purging the first vessel and the second vessel with nitrogen gas while the first vessel and the second vessel cool from a temperature of greater than or equal to 50° C. to room temperature; preparing a solution A, comprising: introducing chromium(III) acetylacetonate and an organic ligand comprising phosphorous and nitrogen to the first vessel, wherein the first vessel is in fluid communication with a Schlenk line; and introducing a toluene solvent to the first vessel via the Schlenk line; preparing a solution B separately from solution A, comprising: introducing dodecyl trimethyl ammonium chloride to the second vessel, wherein the second vessel is in fluid communication with a Schlenk line; and introducing an organoaluminum compound and a toluene solvent to the second vessel via the Schlenk line; introducing solution A and solution B to an ethylene oligomerization reactor, producing the linear alpha olefins, wherein the first vessel and/or the second vessel comprises a lid and the chromium(III) acetylacetonate, the organic ligand, and the dodecyl trimethyl ammonium chloride are introduced to the first vessel and/or the second vessel manually via the lid and under a stream of nitrogen gas.

These and other features and characteristics are more particularly described below.

### BRIEF DESCRIPTION OF THE DRAWINGS

The following is a brief description of the drawings wherein like elements are numbered alike and which are presented for the purposes of illustrating the exemplary embodiments disclosed herein and not for the purposes of limiting the same.

FIG. 1 is a schematic diagram representing a catalyst preparation configuration in a method of producing linear alpha olefins.

FIG. 2 is a schematic diagram representing a catalyst delivery configuration in a method of producing linear alpha olefins.

### DETAILED DESCRIPTION

The method disclosed herein can prepare an ethylene oligomerization catalyst without storage time restrictions or component stability restrictions, and can be used on-demand to produce linear alpha olefins. For example, the method disclosed herein can include preparing a solution A by introducing an organometallic compound and an organic ligand to a first vessel, wherein the first vessel is in fluid communication with a Schlenk line. A solvent can then be introduced to the first vessel via the Schlenk line. The method can further include preparing a solution B separately from solution A, by introducing an ammonium salt to a second vessel, wherein the second vessel is in fluid communication with a Schlenk line. An organoaluminum compound and a solvent can then be introduced to the second vessel via the Schlenk line. The preparation of the two catalyst solutions separate from each other, and under inert operating conditions via a Schlenk line system, allows for on-demand mixing of the solutions (for either immediate or delayed use) and allows for the use of a wider range of catalyst components without typical stability limitations. Linear alpha olefins can then be produced by introducing solution A and solution B to an ethylene oligomerization reactor.

The first and/or second vessel can be in fluid communication with a Schlenk line via a penetrator system. For example, a catalyst solution A can be prepared within a first vessel. The first vessel can then be replaced by a second vessel. A catalyst solution B can then be prepared within the second vessel. The vessels can comprise glass, for example, Pyrex glass. The vessels can comprise a stir plate. For example, the stir plate can control a magnetic stirring bar within the first and/or second vessel. The vessels can also comprise flow meters. The penetrator system can allow quick connections and disconnections. The penetrator system can be, for example, a wellhead and/or packer penetrator.

The vessels can be dried in an oven, for example, at an oven temperature of 50° C. to 110° C., for greater than or equal to 2 hours (including the magnetic stirring bar located within the vessels). The vessels can then be removed from the oven and quickly attached to the Schlenk line while still at the oven temperature. The vessels can then be allowed to cool down to room temperature under vacuum conditions.

The Schlenk line can be sealed and can be used to create safe and inert operating conditions for the preparation of catalyst solutions. For example, the system can be purged with an inert gas, for example, nitrogen gas. This purge sequence can be performed in order to ensure that no moisture or oxygen is present in the system. The purge sequence can occur while the vessels are cooling down from the oven temperature. The inert gas can be supplied by an inert gas tank. An inert gas stream can be passed through the Schlenk line, the penetrator system, and the first and/or second vessel. The Schlenk line can comprise stainless steel. The Schlenk line can further comprise a pressure release valve, a vacuum, a pump, a gas bubbler, or a combination comprising at least one of the foregoing.

In the preparation of a catalyst solution A, an organometallic compound and an organic ligand can be manually introduced to a first vessel. For example, the first vessel can comprise a lid. The lid can be opened and the organometallic compound and the organic ligand can be introduced to the first vessel via the open lid. The manual introduction of components into the first vessel can occur under a stream of inert gas, supplied by the inert gas tank. The organometallic compound and the organic ligand can be introduced to the first vessel while the vessel is still at the oven temperature or after the vessel has cooled.

A solvent can also be introduced to the first vessel. The solvent can comprise an aromatic or aliphatic solvent, or a combination comprising at least one of the foregoing. For example, the solvent can be toluene, benzene, ethylbenzene, cumenene, xylene, mesitylene, hexane, octane, cyclohexane, olefins, such as hexene, heptane, octene, or ethers, such as diethylether or tetrahydrofurane. In an embodiment, the solvent can be an aromatic solvent, for example, toluene. The solvent can be stored in a solvent reservoir. The solvent reservoir can comprise a moisture measuring device. The solvent reservoir can also comprise a drying column with a drying agent. The solvent can be passed from the solvent reservoir to an intermediate solvent reservoir via a solvent stream. The solvent stream can then be passed through the Schlenk line and introduced to the first vessel via the penetrator system. Accordingly, a catalyst solution A can be prepared under inert conditions within the first vessel comprising the organometallic compound, the organic ligand and the solvent. The solution A can be a homogenous liquid.

The organometallic compound can comprise a chromium compound. For example, the chromium compound can be an organic or inorganic salt, a coordination complex, or an organometallic complex of Cr(II) or Cr(III). In some embodiments the chromium compound is CrCl₃(tetrahydrofuran)₃, Cr(III)acetylacetonate, Cr(III)octanoate, chromium hexacarbonyl, Cr(III)-2-ethylhexanoate, benzene(tricarbonyl)-chromium, or Cr(III)chloride. A combination of different chromium compounds can be used. For example, the organometallic compound can comprise chromium (III) acetylacetonate.

The organic ligand can comprise the general structure (A) R₁R₂P—N(R₃)—P(R₄)—N(R₅)—H or (B) R₁R₂P—N(R₃)—P(R₄)—N(R₅)—PR₆R₇, wherein R₁-R₇ are independently selected from halogen, amino, trimethylsilyl, C₁-C₁₀-alkyl, C₆-C₂₀ aryl or any cyclic derivatives of (A) and (B), wherein at least one of the P or N atoms of the PNPN-unit or PNPNP-unit is a member of a ring system, the ring system being formed from one or more constituent compounds of structures (A) or (B) by substitution.

The ligand can include two or more heteroatoms (P, N, O, S, As, Sb, Bi, O, S, or Se) that can be the same or different, wherein the two or more heteroatoms are linked via a linking group. The linking group is a C₁₋₆ hydrocarbylene group or one of the foregoing heteroatoms. Any of the heteroatoms in the ligand can be substituted to satisfy the valence thereof, with a hydrogen, halogen, C₁₋₁₈ hydrocarbyl group, C₁₋₁₀ hydrocarbylene group linked to the same or different heteroatoms to form a heterocyclic structure, amino group of the formula NR^{a}R^{b} wherein each of R^{a} and R^{b} is independently hydrogen or a C₁₋₁₈ hydrocarbyl group, a silyl group of the formula SiR^{a}R^{b}R^{c} wherein each of R^{a}, R^{b}, and R^{c} is independently hydrogen or a C₁₋₁₈ hydrocarbyl group, or a combination comprising at least one of the foregoing substituents. The heteroatoms of the multidentate ligand are preferably a combination comprising phosphorus with nitrogen and sulfur or a combination comprising phosphorous and nitrogen, linked by at least one additional phosphorus or nitrogen heteroatom. In certain embodiments, the ligand can have the backbone PNP, PNPN, NPN, NPNP, NPNPN, PNNP, or cyclic derivatives containing these backbones, wherein one or more of the heteroatoms is linked by a C₁₋₁₀ hydrocarbylene to provide a heterocyclic group. A combination of different ligands can be used.

In some embodiments, the ligand has the backbone PNPNH, which as used herein has the general structure R¹R²P—N(R³)—P(R⁴)—N(R⁵)—H wherein each of R¹, R², R³, R⁴, and R⁵ is independently a hydrogen, halogen, C₁₋₁₈ hydrocarbyl group, amino group of the formula NR^{a}R^{b} wherein each of R^{a} and R^{b} is independently hydrogen or a C₁₋₁₈ hydrocarbyl group, a silyl group of the formula SiR^{a}R^{b}R^{c} wherein each of R^{a}, R^{b}, and R^{c} is independently hydrogen or a C₁₋₁₈ hydrocarbyl group, or two of R¹, R², R³, R⁴, R⁵, R^{a}, or R^{b} taken together are a substituted or unsubstituted C₁₋₁₀ hydrocarbylene group linked to the same or different heteroatoms to form a heterocyclic structure. Exemplary ligands having a heterocyclic structure include the following

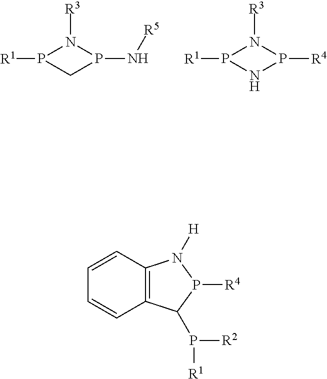

wherein R¹, R², R³, R⁴, R⁵ are as described above. In a specific embodiment, each R¹, R², R³, R⁴, R⁵ are independently hydrogen, substituted or unsubstituted C₁-C₈ alkyl, substituted or unsubstituted C₆-C₂₀ aryl, more preferably unsubstituted C₁-C₆ alkyl or unsubstituted C₆-C₁₀ aryl. A specific example of the ligand is (phenyl)₂PN(iso-propyl)P(phenyl)N(iso-propyl)H, commonly abbreviated Ph₂PN(i-Pr)P(Ph)NH(i-Pr).

A catalyst solution B can be prepared separately from the catalyst solution A (for example, within a second vessel). In the preparation of the catalyst solution B, an ammonium salt can be manually introduced to the second vessel. For example, the ammonium salt can be introduced to the second vessel via the lid. The manual introduction of ammonium salt into the second vessel can occur under a stream of inert gas. A solvent, for example, an aromatic or aliphatic solvent, or a combination comprising at least one of the foregoing can be introduced to the second vessel. For example, the solvent can be toluene, benzene, ethylbenzene, cumenene, xylene, mesitylene, hexane, octane, cyclohexane, olefins, such as hexene, heptane, octene, or ethers, such as diethylether or tetrahydrofurane. In an embodiment, the solvent can be an aromatic solvent, for example, toluene. The solvent can be introduced in the same manner described herein with regards to catalyst solution A.

The ammonium salt can be of the type (H₄E)X, (H₃ER)X, (H₂ER₂)X, (HER₃)X, or (ER₄)X wherein E is N or P, X is Cl, Br, or I, and each R is independently a C₁-C₂₂ hydrocarbyl, preferably a substituted or unsubstituted C₁-C₁₆-alkyl, C₂-C₁₆-acyl, or substituted or unsubstituted C₆-C₂₀-aryl. For example, the ammonium salt can comprise dodecyl trimethyl ammonium chloride.

An organoaluminum compound can also be introduced to the second vessel. For example, the organoaluminum compound can be stored in an organoaluminum reservoir. The organoaluminum compound can be passed from the organoaluminum reservoir to an intermediate organoaluminum reservoir via an organoaluminum stream. The intermediate organoaluminum reservoir can comprise a mass measuring device. The organoaluminum stream can then be passed through the Schlenk line and introduced to the second vessel via the penetrator system. Accordingly, a catalyst solution B can be prepared under inert conditions within the second vessel comprising the organoaluminum compound, the ammonium salt and the solvent. The solution B can be a homogenous liquid.

The organoaluminum compound can be, for example, a tri(C₁-C₆alkyl) aluminum such as triethyl aluminum, (C₁-C₆ alkyl) aluminum sesquichloride, di(C₁-C₆alkyl) aluminum chloride, or (C₁-C₆-alkyl) aluminum dichloride, or an aluminoxane such as methylaluminoxane (MAO). Each alkyl group can be the same or different, and in some embodiments is methyl, ethyl, isopropyl, or isobutyl. For example, the organoaluminum compound can comprise triethylaluminium. A combination of different organoaluminum compounds can also be used.

The type of each component selected for use in the catalyst solutions and relative amount of each component depend on the desired product and desired selectivity. In some embodiments, the concentration of the chromium compound is 0.01 to 100 millimoles per liter (mmol/l), or 0.01 to 10 mmol/l, or 0.01 to 1 mmol/l, or 0.1 to 1.0 mmol/l; and the mole ratio of multidentate ligand:Cr compound:activator is 0.1:1:1 to 10:1:1,000, or 0.5:1:50 to 2:1:500, or 1:1:100 to 5:1:300. Suitable catalyst systems are described, for example, in EP2489431 B1; EP2106854 B1; and WO2004/056479.

The first vessel (comprising catalyst solution A) and the second vessel (comprising catalyst solution B) can be transferred to an on-site location where a reactor is operating. Solution A can be passed to a mixer unit via a dosing pump. For example, the dosing pump can regulate the amount of solution A that is passed to the mixer unit at any given time. Solution B can be passed to the same mixer unit via a second dosing pump. For example, the second dosing pump can regulate the amount of solution B that is passed to the mixer unit. Solution A and solution B can be mixed together within the mixer unit, producing a mixed stream. This mixed stream can then be injected into a reactor, for example, an ethylene oligomerization reactor. The mixed stream can be passed through a third dosing pump. For example, the dosing pump can regulate the amount of mixed catalyst solution that is passed to the ethylene oligomerization reactor. Alternatively, solution A and solution B can be injected directly into the reactor separately, without being mixed.

The reactor can be, for example, a bubble column reactor. A temperature within the reactor can be 10° C. to 100° C., for example, 20° C. to 95° C., for example, 30° C. to 90° C. A pressure within the reactor can be 1,000 kiloPascals to 5,000 kiloPascals, for example, 1,500 kiloPascals to 4,500 kiloPascals, for example, 2,000 kiloPascals to 4,000 kiloPascals.

An oligomerization reaction can occur within the reactor, producing linear alpha olefins. Ethylene oligomerization combines ethylene molecules to produce linear alpha-olefins of various chain lengths with an even number of carbon atoms. This approach results in a distribution of alpha-olefins. Oligomerization of ethylene can produce 1-hexene.

Fischer-Tropsch synthesis to make fuels from synthesis gas derived from coal can recover 1-hexene from the aforementioned fuel streams, where the initial 1-hexene concentration cut can be 60% in a narrow distillation, with the remainder being vinylidenes, linear and branched internal olefins, linear and branched paraffins, alcohols, aldehydes, carboxylic acids, and aromatic compounds. The trimerization of ethylene by homogeneous catalysts has been demonstrated.

There are a wide range of applications for linear alpha olefins. The lower carbon numbers, 1-butene, 1-hexene and 1-octene can be used as comonomers in the production of polyethylene. High density polyethylene (HDPE) and linear low density polyethylene (LLDPE) can use approximately 2-4% and 8-10% of comonomers, respectively.

Another use of C₄-C₈ linear alpha olefins can be for production of linear aldehyde via oxo synthesis (hydroformylation) for later production of short-chain fatty acid, a carboxylic acid, by oxidation of an intermediate aldehyde, or linear alcohols for plasticizer application by hydrogenation of the aldehyde.

An application of 1-decene is in making polyalphaolefin synthetic lubricant base stock (PAO) and to make surfactants in a blend with higher linear alpha olefins.

C₁₀-C₁₄ linear alpha olefins can be used in making surfactants for aqueous detergent formulations. These carbon numbers can be reacted with benzene to make linear alkyl benzene (LAB), which can be further sulfonated to linear alkyl benzene sulfonate (LABS), a popular relatively low cost surfactant for household and industrial detergent applications.

Although some C₁₄ alpha olefin can be sold into aqueous detergent applications, C₁₄ has other applications such as being converted into chloroparaffins. A recent application of C₁₄ is on-land drilling fluid base stock, replacing diesel or kerosene in that application. Although C₁₄ is more expensive than middle distillates, it has a significant advantage environmentally, being much more biodegradable and in handling the material, being much less irritating to skin and less toxic.

C₁₆-C₁₈ linear olefins find their primary application as the hydrophobes in oil-soluble surfactants and as lubricating fluids themselves. C₁₆-C₁₈ alpha or internal olefins are used as synthetic drilling fluid base for high value, primarily off-shore synthetic drilling fluids. The preferred materials for the synthetic drilling fluid application are linear internal olefins, which are primarily made by isomerizing linear alpha-olefins to an internal position. The higher internal olefins appear to form a more lubricious layer at the metal surface and are recognized as a better lubricant. Another application for C₁₆-C₁₈ olefins is in paper sizing. Linear alpha olefins are, once again, isomerized into linear internal olefins and are then reacted with maleic anhydride to make an alkyl succinic anhydride (ASA), a popular paper sizing chemical.

C₂₀-C₃₀ linear alpha olefins production capacity can be 5-10% of the total production of a linear alpha olefin plant. These are used in a number of reactive and non-reactive applications, including as feedstocks to make heavy linear alkyl benzene (LAB) and low molecular weight polymers used to enhance properties of waxes.

The use of 1-hexene can be as a comonomer in production of polyethylene. High-density polyethylene (HDPE) and linear low-density polyethylene (LLDPE) use approximately 2-4% and 8-10% of comonomers, respectively.

Another use of 1-hexene is the production of the linear aldehyde heptanal via hydroformylation (oxo synthesis). Heptanal can be converted to the short-chain fatty acid heptanoic acid or the alcohol heptanol.

A more complete understanding of the components, processes, and apparatuses disclosed herein can be obtained by reference to the accompanying drawings. These figures (also referred to herein as “FIG.”) are merely schematic representations based on convenience and the ease of demonstrating the present disclosure, and are, therefore, not intended to indicate relative size and dimensions of the devices or components thereof and/or to define or limit the scope of the exemplary embodiments. Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

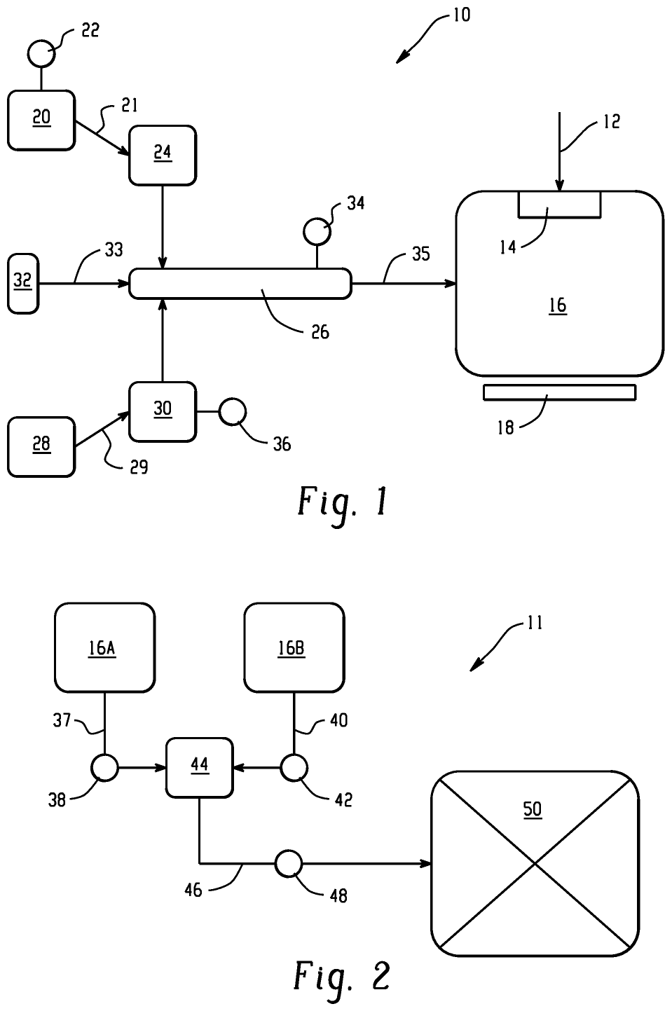

Referring now to FIG. 1, this schematic diagram represents a catalyst preparation configuration 10 in a method of producing linear alpha olefins. The configuration 10 can comprise a vessel 16 in fluid communication with a Schlenk line 26 via a penetrator system 35. For example, a catalyst solution A can be prepared within a first vessel 16. The first vessel 16 can then be replaced by a second vessel 16. A catalyst solution B can then be prepared within the second vessel 16. The vessel 16 can comprise a stir plate 18.

The Schlenk line 26 can be sealed and can be used to create inert operating conditions for the catalyst preparation configuration 10. For example, the configuration 10 can be purged with an inert gas. The inert gas can be supplied by the inert gas tank 32. The inert gas can be passed through the Schlenk line 26 and the first vessel 16 via the inert gas stream 33 and the penetrator system 35. The Schlenk line 26 can further comprise a pressure release valve 34.

In the preparation of a catalyst solution A, an organometallic compound and an organic ligand can be manually introduced (represented by element 12) to a first vessel 16. For example, the first vessel 16 can comprise a lid 14. The lid 14 can be opened and the organometallic compound and the organic ligand can be introduced to the first vessel 16 via the open lid 14. The manual introduction 12 of components into the first vessel 16 can occur under a stream of inert gas, supplied by inert gas tank 32.

A solvent can also be introduced to the first vessel 16. For example, solvent can be stored in a solvent reservoir 20. The solvent reservoir 20 can comprise a moisture measuring device 22. The solvent can be passed from the solvent reservoir 20 to an intermediate solvent reservoir 24 via a solvent stream 21. The solvent stream 21 can then be passed through the Schlenk line 26 and introduced to the first vessel 16 via the penetrator system 35. Accordingly, a catalyst solution A can be prepared under inert conditions within the first vessel 16 comprising the organometallic compound, the organic ligand and the solvent.

A catalyst solution B can be prepared separately from the catalyst solution A (for example, within a second vessel 16). In the preparation of the catalyst solution B, an ammonium salt can be manually introduced (represented by element 12) to the second vessel 16. For example, the ammonium salt can be introduced to the second vessel 16 via the lid 14. The manual introduction 12 of ammonium salt into the second vessel 16 can occur under a stream of inert gas, supplied by inert gas tank 32. A solvent can be introduced to the second vessel 16. For example, the solvent can be introduced in the same manner described herein with regards to catalyst solution A.

An organoaluminum compound can also be introduced to the second vessel 16. For example, the organoaluminum compound can be stored in an organoaluminum reservoir 28. The organoaluminum compound can be passed from the organoaluminum reservoir 28 to an intermediate organoaluminum reservoir 30 via an organoaluminum stream 29. The intermediate organoaluminum reservoir 30 can comprise a mass measuring device 36. The organoaluminum stream 29 can then be passed through the Schlenk line 26 and introduced to the second vessel 16 via the penetrator system 35. Accordingly, a catalyst solution B can be prepared under inert conditions within the second vessel 16 comprising the organoaluminum compound, the ammonium salt and the solvent.

Referring now to FIG. 2, this schematic diagram represents a catalyst delivery configuration 11 in a method of producing linear alpha olefins. This configuration 11 can comprise a first vessel 16A (comprising catalyst solution A) and a second vessel 16B (comprising catalyst solution B).

Solution A can be passed to a mixer unit 44 via a stream 37. The stream 37 can be passed through a dosing pump 38. For example, the dosing pump 38 can regulate the amount of solution A that is passed to the mixer unit 44 at any given time. Solution B can be passed to the mixer unit 44 via a stream 40. The stream 40 can be passed through a dosing pump 42. For example, the dosing pump 42 can regulate the amount of solution B that is passed to the mixer unit 44.

Solution A and solution B can be mixed together within the mixer unit 44, producing a mixed stream 46. This mixed stream 46 can then be injected into a reactor 50, for example, an ethylene oligomerization reactor 50. The mixed stream 46 can be passed through a dosing pump 48. For example, the dosing pump 48 can regulate the amount of mixed catalyst solution that is passed to the ethylene oligomerization reactor 50. An oligomerization reaction can occur within the reactor 50, producing linear alpha olefins.

The following examples are merely illustrative of the method of producing linear alpha olefins disclosed herein and is not intended to limit the scope hereof.

### EXAMPLES

Trials were conducted in accordance with the catalyst preparation method disclosed herein (as depicted in FIG. 1). The total volume of each solution prepared was 4 liters. All operations were conducted under an inert atmosphere of nitrogen gas using a stainless steel Schlenk line. The vessels used were dried in an oven at 50° C. to 110° C. for at least 2 hours (including a magnetic stirring bar located within the vessels). The vessels were then removed from the oven and quickly attached to the Schlenk line while still at the oven temperature. The vessels were attached to the Schlenk line via a penetrator system and allowed to cool down to room temperature under vacuum conditions. A nitrogen purge sequence was then performed while the vessel was cooling down in order to ensure that no moisture or oxygen was present in the system.

### Example 1

A catalyst solution A was prepared in accordance with the catalyst preparation method disclosed herein (as depicted in FIG. 1). At room temperature, the lid of the first vessel was opened under a stream of nitrogen. 30 grams (g) of chromium (III) acetylacetonate and 42 g of a ligand (a phosphorous and nitrogen compound) were pre-weighed in a nitrogen filled glove-box and then introduced to the first vessel via the open lid. A nitrogen purge sequence was then performed in order to ensure that no moisture or oxygen was present in the system. The total amount of toluene solvent in the intermediate solvent reservoir (4 liters) was then added to the first vessel via the Schlenk line and stirred until a homogeneous solution is obtained. The first vessel (containing the prepared solution A) was then disconnected from the Schlenk line via the penetrator system.

### Example 2

A catalyst solution B was prepared in accordance with the catalyst preparation method disclosed herein (as depicted in FIG. 1). At room temperature, the lid of the second vessel was opened under a stream of nitrogen. 180 g of Dodecyl trimethyl ammonium chloride was pre-weighed in a nitrogen filled glove-box and then introduced to the second vessel via the open lid. 60% to 70% of the 4 liters of toluene stored in the intermediate solvent reservoir was then added to the second vessel via the Schlenk line and stirred until a fine suspension was obtained. 251 g triethylaluminium was weighed in the intermediate organoaluminum reservoir and then slowly transferred into the second vessel via the Schlenk line. The solution B was then stirred via differential nitrogen pressure until a clear and homogeneous mixture was obtained. The remaining solvent in the intermediate solvent reservoir was then transferred into the second vessel and stirred for an additional 10 minutes. The second vessel (containing the prepared solution B) was then disconnected from the Schlenk line via the penetrator system.

The methods disclosed herein include(s) at least the following aspects:

Aspect 1: A method of producing linear alpha olefins, comprising: preparing a solution A, comprising: introducing an organometallic compound and an organic ligand to a first vessel, wherein the first vessel is in fluid communication with a Schlenk line; and introducing a solvent to the first vessel via the Schlenk line; preparing a solution B separately from solution A, comprising: introducing an ammonium salt to a second vessel, wherein the second vessel is in fluid communication with a Schlenk line; and introducing an organoaluminum compound and a solvent to the second vessel via the Schlenk line; producing the linear alpha olefins by introducing solution A and solution B to an ethylene oligomerization reactor.

Aspect 2: The method of Aspect 1, further comprising drying the first vessel and/or the second vessel prior to preparing solution A and/or solution B, preferably, wherein the drying occurs in an oven at a temperature greater than or equal to 50° C.

Aspect 3: The method of any of the preceding aspects, further comprising purging the first vessel and/or the second vessel with an inert gas, preferably, wherein the inert gas is nitrogen.

Aspect 4: The method of Aspect 3, wherein the purging occurs while the first vessel and/or the second vessel cools from a temperature of greater than or equal to 50° C. to room temperature.

Aspect 5: The method of any of the preceding aspects, wherein the first vessel and/or the second vessel comprises glass.

Aspect 6: The method of any of the preceding aspects, further comprising mixing solution A and solution B before introduction to the ethylene oligomerization reactor.

Aspect 7: The method of any of the preceding aspects, wherein the Schlenk line comprises stainless steel.

Aspect 8: The method of any of the preceding aspects, wherein solution A and/or solution B are a homogenous liquid.

Aspect 9: The method of any of the preceding aspects, wherein the solvent comprises an aromatic or aliphatic solvent, or a combination comprising at least one of the foregoing, preferably toluene, benzene, ethylbenzene, cumenene, xylene, mesitylene, hexane, octane, cyclohexane, olefins, such as hexene, heptane, octene, or ethers, such as diethylether or tetrahydrofurane, more preferably an aromatic solvent, most preferably toluene.

Aspect 10: The method of any of the preceding aspects, wherein the first vessel and/or the second vessel comprises a stir plate and/or a flow meter.

Aspect 11: The method of any of the preceding aspects, wherein the solvent is passed from a solvent reservoir to an intermediate solvent reservoir, and then to the first vessel and/or the second vessel via the Schlenk line; preferably, wherein the solvent reservoir comprises a drying column and/or a moisture measuring device.

Aspect 12: The method of any of the preceding aspects, wherein the organoaluminum compound is passed from an organoaluminum reservoir to an intermediate organoaluminum reservoir, and then to the first vessel and/or the second vessel via the Schlenk line, preferably, wherein the intermediate organoaluminum reservoir comprises a mass measuring device.

Aspect 13: The method of any of the preceding aspects, wherein the Schlenk line comprises a pump, a pressure release valve, an inert gas tank, a vacuum, or a combination comprising at least one of the foregoing.

Aspect 14: The method of any of the preceding aspects, wherein the first vessel and/or the second vessel is in fluid communication with the Schlenk line via a penetrator system.

Aspect 15: The method of any of the preceding aspects, wherein the organometallic compound comprises chromium (III) acetylacetonate.

Aspect 16: The method of any of the preceding aspects, wherein the organic ligand comprises phosphorous and nitrogen.

Aspect 17: The method of any of the proceeding aspects, wherein the ammonium salt comprises dodecyl trimethyl ammonium chloride.

Aspect 18: The method of any of the preceding aspects, wherein the organoaluminum compound comprises triethylaluminium.

Aspect 19: The method of any of the preceding aspects, wherein the first vessel and/or the second vessel comprises a lid and the organometallic compound, the organic ligand, the ammonium salt, or a combination comprising at least one of the foregoing is introduced to the first vessel and/or the second vessel manually via the lid and under a stream of inert gas.

Embodiment 20: A method of producing linear alpha olefins, comprising: drying a first vessel and a second vessel in an oven at a temperature greater than or equal to 50° C.; purging the first vessel and the second vessel with nitrogen gas while the first vessel and the second vessel cool from a temperature of greater than or equal to 50° C. to room temperature; preparing a solution A, comprising: introducing chromium(III) acetylacetonate and an organic ligand comprising phosphorous and nitrogen to the first vessel, wherein the first vessel is in fluid communication with a Schlenk line; and introducing a toluene solvent to the first vessel via the Schlenk line; preparing a solution B separately from solution A, comprising: introducing dodecyl trimethyl ammonium chloride to the second vessel, wherein the second vessel is in fluid communication with a Schlenk line; and introducing an organoaluminum compound and a toluene solvent to the second vessel via the Schlenk line; introducing solution A and solution B to an ethylene oligomerization reactor, producing the linear alpha olefins, wherein the first vessel and/or the second vessel comprises a lid and the chromium(III) acetylacetonate, the organic ligand, and the dodecyl trimethyl ammonium chloride are introduced to the first vessel and/or the second vessel manually via the lid and under a stream of nitrogen gas.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention. The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.). Disclosure of a narrower range or more specific group in addition to a broader range is not a disclaimer of the broader range or larger group. “Combination” is inclusive of blends, mixtures, alloys, reaction products, and the like. Furthermore, the terms “first,” “second,” and the like, herein do not denote any order, quantity, or importance, but rather are used to denote one element from another. The terms “a” and “an” and “the” herein do not denote a limitation of quantity, and are to be construed to cover both the singular and the plural, unless otherwise indicated herein or clearly contradicted by context. “Or” means “and/or.” The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

The modifier “about” used in connection with a quantity is inclusive of the stated value and has the meaning dictated by the context (e.g., includes the degree of error associated with measurement of the particular quantity). The notation “±10%” means that the indicated measurement can be from an amount that is minus 10% to an amount that is plus 10% of the stated value. The terms “front”, “back”, “bottom”, and/or “top” are used herein, unless otherwise noted, merely for convenience of description, and are not limited to any one position or spatial orientation. “Optional” or “optionally” means that the subsequently described event or circumstance can or cannot occur, and that the description includes instances where the event occurs and instances where it does not. Unless defined otherwise, technical and scientific terms used herein have the same meaning as is commonly understood by one of skill in the art to which this invention belongs. A “combination” is inclusive of blends, mixtures, alloys, reaction products, and the like.

All cited patents, patent applications, and other references are incorporated herein by reference in their entirety. However, if a term in the present application contradicts or conflicts with a term in the incorporated reference, the term from the present application takes precedence over the conflicting term from the incorporated reference

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

What is claimed is:

1. A method of producing linear alpha olefins, comprising:
preparing a solution A, comprising:
introducing an organometallic compound and an organic ligand to a first vessel,
wherein the first vessel is in fluid communication with a Schlenk line; and
introducing a solvent to the first vessel via the Schlenk line;
preparing a solution B separately from solution A, comprising:
introducing an ammonium salt to a second vessel, wherein the second vessel is in fluid communication with a Schlenk line; and
introducing an organoaluminum compound and a solvent to the second vessel via the Schlenk line;
producing the linear alpha olefins by introducing solution A and solution B to an ethylene oligomerization reactor.

2. The method of claim 1, further comprising drying the first vessel and/or the second vessel prior to preparing solution A and/or solution B, preferably, wherein the drying occurs in an oven at a temperature greater than or equal to 50° C.

3. The method of claim 1, further comprising purging the first vessel and/or the second vessel with an inert gas, preferably, wherein the inert gas is nitrogen.

4. The method of claim 3, wherein the purging occurs while the first vessel and/or the second vessel cools from a temperature of greater than or equal to 50° C. to room temperature.

5. The method of claim 1, wherein the first vessel and/or the second vessel comprises glass.

6. The method of claim 1, further comprising mixing solution A and solution B before introduction to the ethylene oligomerization reactor.

7. The method of claim 1, wherein the Schlenk line comprises stainless steel.

8. The method of claim 1, wherein solution A and/or solution B are a homogenous liquid.

9. The method of claim 1, wherein the solvent comprises an aromatic or aliphatic solvent, or a combination comprising at least one of the foregoing, preferably toluene, benzene, ethylbenzene, cumenene, xylene, mesitylene, hexane, octane, cyclohexane, olefins, such as hexene, heptane, octene, or ethers, such as diethylether or tetrahydrofurane, more preferably an aromatic solvent, most preferably toluene.

10. The method of claim 1, wherein the first vessel and/or the second vessel comprises a stir plate and/or a flow meter.

11. The method of claim 1, wherein the solvent is passed from a solvent reservoir to an intermediate solvent reservoir, and then to the first vessel and/or the second vessel via the Schlenk line; preferably, wherein the solvent reservoir comprises a drying column and/or a moisture measuring device.

12. The method of claim 1, wherein the organoaluminum compound is passed from an organoaluminum reservoir to an intermediate organoaluminum reservoir, and then to the first vessel and/or the second vessel via the Schlenk line, preferably, wherein the intermediate organoaluminum reservoir comprises a mass measuring device.

13. The method of claim 1, wherein the Schlenk line comprises a pump, a pressure release valve, an inert gas tank, a vacuum, or a combination comprising at least one of the foregoing.

14. The method of claim 1, wherein the first vessel and/or the second vessel is in fluid communication with the Schlenk line via a penetrator system.

15. The method of claim 1, wherein the organometallic compound comprises chromium (III) acetylacetonate.

16. The method of claim 1, wherein the organic ligand comprises phosphorous and nitrogen.

17. The method of claim 1, wherein the ammonium salt comprises dodecyl trimethyl ammonium chloride.

18. The method of claim 1, wherein the organoaluminum compound comprises triethylaluminium.

19. The method of claim 1, wherein the first vessel and/or the second vessel comprises a lid and the organometallic compound, the organic ligand, the ammonium salt, or a combination comprising at least one of the foregoing is introduced to the first vessel and/or the second vessel manually via the lid and under a stream of inert gas.

20. A method of producing linear alpha olefins, comprising:
drying a first vessel and a second vessel in an oven at a temperature greater than or equal to 50° C.;
purging the first vessel and the second vessel with nitrogen gas while the first vessel and the second vessel cool from a temperature of greater than or equal to 50° C. to room temperature;
preparing a solution A, comprising:
introducing chromium(III) acetylacetonate and an organic ligand comprising phosphorous and nitrogen to the first vessel, wherein the first vessel is in fluid communication with a Schlenk line; and
introducing a toluene solvent to the first vessel via the Schlenk line;
preparing a solution B separately from solution A, comprising:
introducing dodecyl trimethyl ammonium chloride to the second vessel, wherein the second vessel is in fluid communication with a Schlenk line; and
introducing an organoaluminum compound and a toluene solvent to the second vessel via the Schlenk line;
introducing solution A and solution B to an ethylene oligomerization reactor, producing the linear alpha olefins, wherein the first vessel and/or the second vessel comprises a lid and the chromium(III) acetylacetonate, the organic ligand, and the dodecyl trimethyl ammonium chloride are introduced to the first vessel and/or the second vessel manually via the lid and under a stream of nitrogen gas.
